(19) United States
(12) Patent Application Publication
(10) Pub. No.: US 2020/0051148 A1
(43) Pub. Date: Feb. 13, 2020

(54) AUTONOMOUSLY VERIFYING LEGAL RESTRICTIONS AND VENDING OF ALCOHOLIC BEVERAGES

(71) Applicant: Anheuser-Busch Companies, LLC, St. Louis, MO (US)

(72) Inventors: Andrew Green, New York, NY (US); Andrew Smith, St. Louis, MO (US)

(21) Appl. No.: 16/535,876

(22) Filed: Aug. 8, 2019

Related U.S. Application Data
(60) Provisional application No. 62/717,327, filed on Aug. 10, 2018.

(51) Int. Cl. G06Q 30/06; G06K 9/00; G01N 33/49

(52) U.S. Cl. CPC G06Q 30/0607; G01N 33/49; G06K 9/00087; G06K 9/00013

(57) ABSTRACT

Autonomous selling, vending, and/or distributing of alcoholic beverages upon autonomous verification that the prospective purchaser or consumer satisfies any applicable age restrictions and/or is not intoxicated beyond the acceptable limit.

### CROSS-REFERENCE TO RELATED APPLICATIONS

This application claims the benefit of U.S. Provisional Application No. 62/717,327, filed Aug. 10, 2018, which is hereby incorporated by reference in its entirety.

### FIELD

The field relates to automated vending of alcoholic beverages, and more particularly to systems and methods for autonomously verifying that a prospective purchaser or consumer of an alcoholic beverage satisfies legal restrictions on the purchase and autonomously vending the alcoholic beverage based upon the legal restriction verification.

### BACKGROUND

Laws or regulations in many jurisdictions throughout the world restrict the sale or distribution of alcoholic beverages. One such restriction is a legal drinking age, which prohibits the purchase and possession of alcoholic beverages by minors. For example, states of the United States restrict the purchase and possession of alcoholic beverages to those who are 21 years of age or older. Another restriction is a prohibition against selling or distributing alcoholic beverages to an intoxicated person. Because of these restrictions, selling or distributing alcoholic beverages typically requires a human to manually verify that the prospective purchaser or consumer meets the legal drinking age and is not intoxicated beyond an acceptable limit at the time of purchase.

However, reliance on human verification is not always desirable. For example, human verification of the age of a prospective purchaser or consumer via a photo identification card can take around 10 seconds or more in some instances and can be susceptible to forgery. A disadvantage of relying on a human to determine whether or not a prospective purchaser or consumer is intoxicated is the subjective nature of the manual determination. For instance, a prospective purchaser or consumer may naturally exhibit characteristics that a human verifier mistakes for being indicative of the prospective purchaser or consumer being intoxicated, or the prospective purchaser or consumer may be able to suppress characteristics of intoxication to fool the human verifier. Yet another potential disadvantage of relying on humans in some instances is the physical space and time required for the human to transition between legal restriction verification activities, alcoholic beverage retrieval activities, and payment transaction activities.

### SUMMARY

Systems and methods that autonomously sell, vend, and/or distribute alcoholic beverages upon autonomously verifying that the prospective purchaser or consumer satisfies any applicable age restrictions and/or is not intoxicated beyond the acceptable limit.

In an example embodiment, a system includes a sensor, an age verifier, an intoxication verifier, and an alcoholic beverage dispenser. The sensor is operable to receive biometric information of a prospective alcoholic beverage purchaser or consumer and blood alcohol content (BAC) information of the prospective purchaser or consumer. The age verifier is operable to autonomously determine an age of the prospective purchaser or consumer based at least in part upon the received biometric information and autonomously determine whether the determined age of the prospective purchaser or consumer satisfies a minimum alcoholic beverage purchasing age. The intoxication verifier is operable to autonomously determine an intoxication level of the prospective purchaser or consumer based at least in part upon the received BAC information and autonomously determine whether the determined intoxication level satisfies an intoxication level threshold. The alcoholic beverage dispenser is operable to autonomously vend or dispense one or more alcoholic beverages to the purchaser or consumer based at least in part upon autonomously determining that the prospective purchaser or consumer satisfies the minimum alcoholic beverage purchasing age and the intoxication level threshold.

In another example embodiment, a system includes a sensor, an age verifier, an intoxication verifier, and an alcoholic beverage dispenser. The sensor is operable to receive biometric information of a prospective alcoholic beverage purchaser or consumer or blood alcohol content (BAC) information of the prospective purchaser or consumer. The age verifier is operable to autonomously determine an age of the prospective purchaser or consumer based at least in part upon the received biometric information and autonomously determine whether the determined age of the prospective purchaser or consumer satisfies a minimum alcoholic beverage purchasing age. The intoxication verifier is operable to autonomously determine an intoxication level of the prospective purchaser or consumer based at least in part upon the received BAC information and autonomously determine whether the determined intoxication level satisfies an intoxication level threshold. The alcoholic beverage dispenser is operable to autonomously vend or dispense one or more alcoholic beverages to the purchaser or consumer based at least in part upon autonomously determining that the prospective purchaser or consumer satisfies either the minimum alcoholic beverage purchasing age or the intoxication level threshold.

### BRIEF DESCRIPTION OF THE DRAWINGS

FIG. 1 illustrates a block diagram of an exemplary system according to an embodiment of the present invention.

FIGS. 2A-2C each illustrate a flowchart of a process for autonomously selling, vending, and/or distributing alcoholic beverages according to embodiments of the present invention.

FIG. 3 illustrates a flowchart of a process for autonomously verifying that a prospective purchaser or consumer of alcoholic beverages satisfies any applicable age restrictions according to an embodiment of the present invention.

FIG. 4 illustrates a flowchart of a process for autonomously verifying that a prospective purchaser or consumer of alcoholic beverages is not intoxicated beyond the acceptable limit according to an embodiment of the present invention.

FIG. 5 illustrates a block diagram of another exemplary system according to an embodiment of the present invention.

FIG. 6 illustrates a flowchart of a process for autonomously selling, vending, and/or distributing alcoholic beverages, according to another exemplary embodiment.

### DETAILED DESCRIPTION

A platform to enable autonomous (i.e., without a human) selling, vending, and/or distributing of legally restricted beverages while verifying the prospective purchaser or consumer satisfies the legal restrictions is described herein. In an aspect, the platform enables autonomous selling, vending, and/or distributing of alcoholic beverages (e.g., beer, cider, wine, spirits, liqueurs, etc.) while preventing underage drinking and over consumption. In order to autonomously sell, vend, and/or distribute alcoholic beverages, the platform is configured to verify at least one of two things: 1) the prospective purchaser or consumer satisfies any applicable age restrictions (e.g., is 21 or older, etc.) and 2) the prospective purchaser or consumer is not intoxicated beyond the acceptable limit per the specific use case. In an embodiment, satisfaction of applicable age restrictions is verified by an identity database that allows users to create biometric profiles attaching their identity to their unique fingerprints. Moreover, verification that the prospective purchaser or consumer is not intoxicated beyond the acceptable limit is accomplished via biometric technology that can accurately assess blood alcohol content through spectroscopic scans of the finger. By integrating both of these technologies into the platform, systems and methods in accordance with this disclosure enable several new use cases for selling, vending, and/or distributing alcoholic beverages including, but not limited to, vending machines, self-checkout, click and collect, ecommerce deliveries, and more. For example, applications of this platform include vending machines in sports stadium and airports, where the integrated platform will allow consumers to purchase alcoholic beverages from a vending machine via simple finger print scans without the concerns of underage drinking or overconsumption.

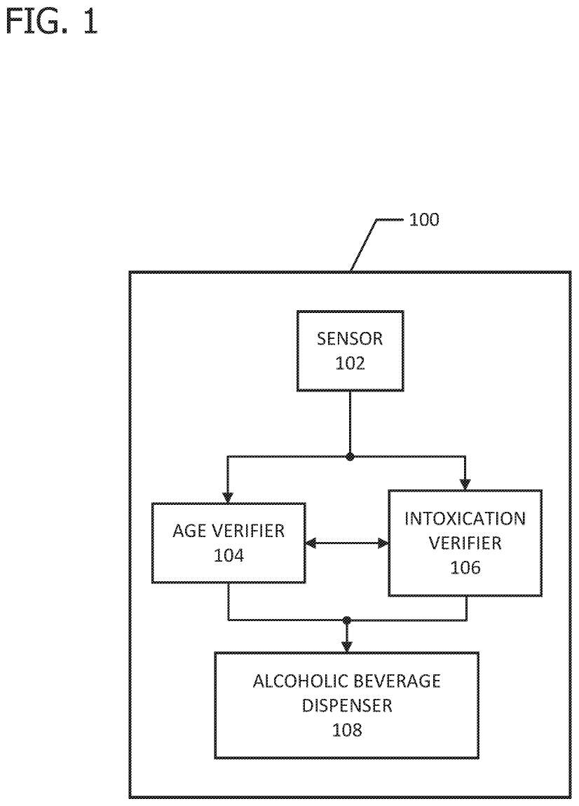

FIG. 1 is a block diagram of a system 100, according an exemplary embodiment. The system 100 includes at least a sensor 102, an age verifier 104, an intoxication verifier 106, and an alcoholic beverage dispenser 108.

The sensor 102 is operable to receive information about physiological or behavioral characteristics of a prospective alcoholic beverage purchaser or consumer for the purpose of identity verification and/or information about the blood alcohol content (BAC) of the prospective purchaser or consumer. The sensor 102 may comprise at least a biometric device and/or a non-invasive blood alcohol measurement device. In an exemplary embodiment, the sensor 102 is a non-invasive sensor operable to scan a finger, including fingerprint, of the prospective purchaser or consumer. In this exemplary embodiment, the sensor 102 may include a fingerprint reader (e.g., solid-state fingerprint reader, optical fingerprint reader, non-contact/touchless fingerprint reader, etc.), a blackbody light source operable to illuminate the skin of the finger, an interferometer operable to filter light reflected by the skin for wavelengths relevant to blood alcohol content measurement, and a detector (e.g., indium gallium arsenide detector, etc.) operable to analyze the filtered light and report a BAC level of the prospective purchaser or consumer.

One of ordinary skill in the art will understand that other biometric devices are within the scope of the invention including, but not limited to, chemical biometric devices (e.g., DNA analysis, etc.), visual biometric devices (e.g., face recognition, fingerprint recognition, retina recognition, etc.), behavioral biometric devices (e.g., gait signatures, etc.), olfactory biometric devices (e.g., odor signatures, etc.), auditory biometric devices (e.g., voice recognition), and combinations thereof. Moreover, one of ordinary skill in the art will understand that other BAC measurement devices and techniques are within the scope of the invention including, but not limited to, blood analysis, breath analysis, urine analysis, and saliva analysis.

The age verifier 104 is operable to receive information about the identity of a prospective purchaser or consumer. In an exemplary embodiment, the age verifier 104 receives information about the identity of the prospective purchaser or consumer from sensor 102. The age verifier 104 may use the received information to look up identification information about the prospective purchaser or consumer and verify an age of the prospective purchaser or consumer. In an embodiment, the identification information may be stored in a database. The database may be stored locally on system 100 (e.g., in one or more memory devices, etc.). In an alternative embodiment, the database is stored remotely from system 100 and system 100 utilizes a telecommunications network (e.g., the Internet, etc.) to access the database. The database may be provided by an entity associated with the manufacture or distribution of system 100, a government entity, a third-party private entity, or any combination thereof. In an exemplary embodiment, age verifier 104 returns an integer representing the verified age of the prospective purchaser or consumer to one or more other components of system 100 for that component to determine whether the verified age satisfies the minimum alcoholic beverage purchasing age. For example, the minimum alcoholic beverage purchasing age may be tied to a legal jurisdiction in which system 100 is located. In another exemplary embodiment, age verifier 104 determines whether the verified age satisfies the minimum alcohol purchasing age and returns a binary indication (e.g., YES/NO, TRUE/FALSE, etc.) of the determination to one or more other components of system 100.

In an exemplary embodiment, the age verifier 104 includes a camera operable to obtain an image of a finger of the prospective purchaser or consumer and a communication network connection to a server computing device including a database operable to verify the identity and age of the prospective purchaser or consumer. In an exemplary embodiment, age verifier 104 can be embodied, at least in part, as processor-executable instructions stored on tangible, non-transitory memory devices and comprising application programs, program modules, routines, objects, data structures, functions, or the like.

The intoxication verifier 106 is operable to receive information about the BAC of the prospective purchaser or consumer. In an exemplary embodiment, the intoxication verifier 106 receives information about the BAC of the prospective purchaser or consumer from sensor 102. The intoxication verifier 106 is operable to verify an intoxication level of the prospective purchaser or consumer based on the received information and compare the intoxication level to an intoxication level threshold. For example, the intoxication verifier 106 may store the intoxication level threshold, compare the received BAC information to the intoxication level threshold, and return an objective determination of the intoxication of the prospective purchaser or consumer. The intoxication level threshold may be predefined or dynamic in some embodiments. For example, the intoxication level threshold may be set to legal limits at which point operation of motor vehicles is prohibited (e.g., 0.08% in the United States) or may be altered depending upon other factors. In an exemplary embodiment, the intoxication verifier 106 returns a value representing the verified intoxication level of the prospective purchaser or consumer to one or more other components of system 100 for that component to determine whether the verified intoxication level exceeds the intoxication level threshold. In another exemplary embodiment, the intoxication verifier 106 determines whether the verified intoxication level exceeds the intoxication level threshold and returns a binary indication (e.g., YES/NO, TRUE/FALSE, etc.) of the determination to one or more other components of system 100.

In an exemplary embodiment, the intoxication verifier 106 includes a blackbody light source operable to illuminate the skin of the finger of the prospective purchaser or consumer, an interferometer operable to filter light reflected by the skin for wavelengths relevant to BAC measurement, and a detector (e.g., indium gallium arsenide detector, etc.) operable to analyze the filtered light and report the BAC level of the prospective purchaser or consumer. One of ordinary skill in the art will understand that other spectroscopic BAC measurement devices and techniques are within the scope of the invention. In an exemplary embodiment, intoxication verifier 106 can be embodied, at least in part, as processor-executable instructions stored on tangible, non-transitory memory devices and comprising application programs, program modules, routines, objects, data structures, functions, or the like.

The alcoholic beverage dispenser 108 is operable to autonomously vend or dispense one or more alcoholic beverages to the purchaser or consumer based at least in part upon autonomous verification that the purchaser or consumer satisfies the minimum alcohol purchasing age and does not exceed the intoxication level threshold. The alcoholic beverage dispenser 108 may be any dispenser or vending machine operable to dispense or vend beverages including, but not limited to, machines operable to vend containers that already contain beverages (e.g., cans, bottles, etc.), machines operable to vend containers and subsequently vend beverages into the vended container, and machines operable to vend beverages into a container placed by the purchaser or consumer. For example, the alcoholic beverage dispenser 108 may include a can dispenser, a bottle dispenser, a beer tap, a soft drink dispenser and a distilled spirits dispenser (e.g., for mixed drinks), and the like.

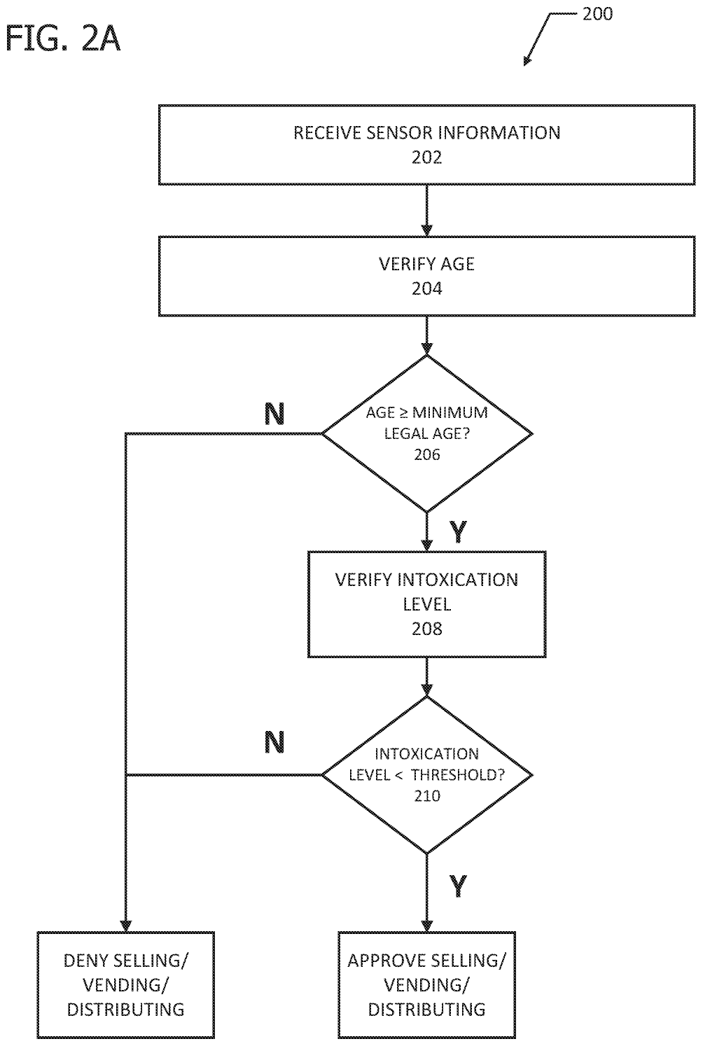

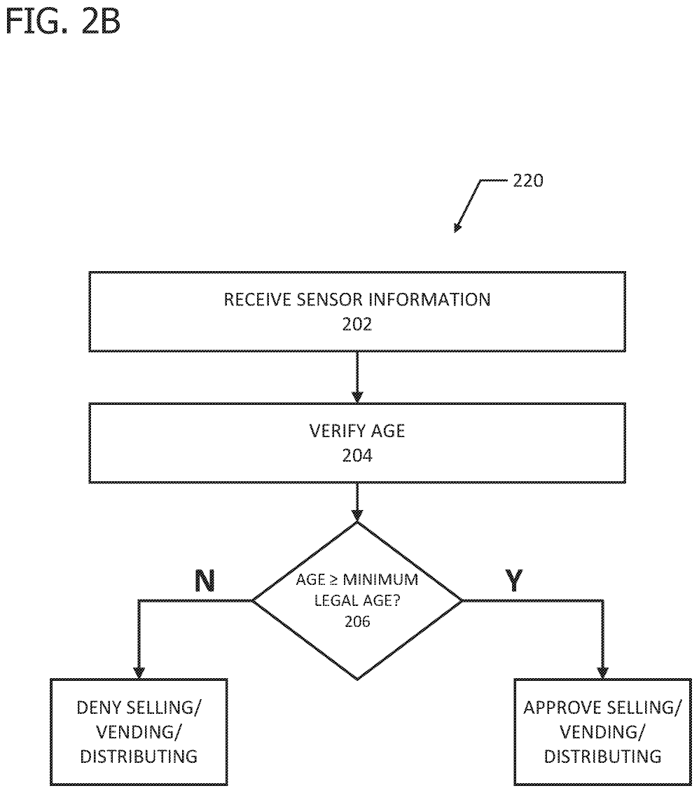

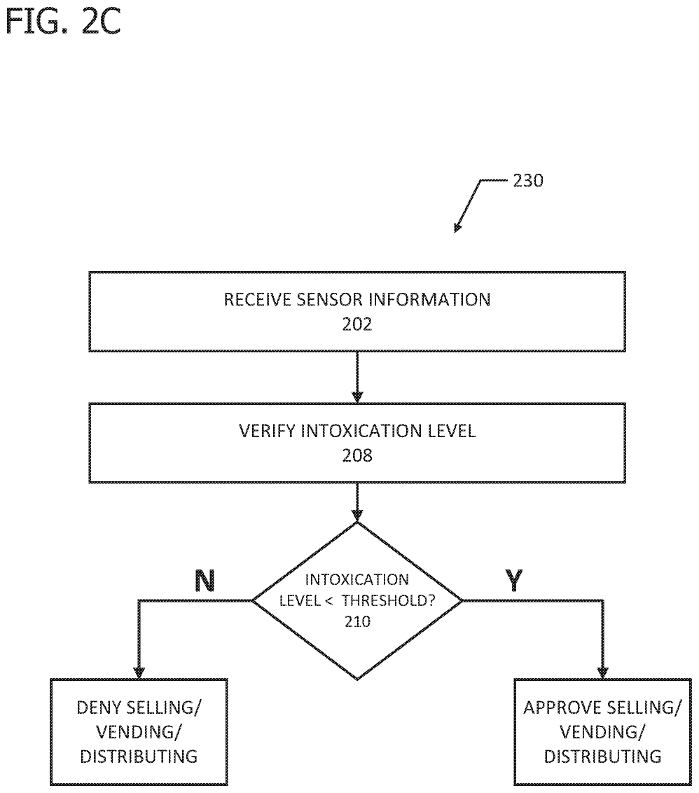

FIG. 2A illustrates a method 200 of autonomously selling, vending, and/or distributing alcoholic beverages, according to an exemplary embodiment. At 202, information about physiological or behavioral characteristics of a prospective alcoholic beverage purchaser or consumer and/or information about the blood alcohol content (BAC) of the prospective purchaser or consumer is received. Using the received information, the age of the prospective purchaser or consumer is verified at 204. At 206, it is determined whether the verified age of the prospective purchaser or consumer is equal to or greater than a minimum alcoholic beverage purchasing age. When the verified age of the prospective purchaser or consumer is less than the minimum alcoholic beverage purchasing age the method 200 denies selling, vending, and/or distributing alcoholic beverages to the prospective purchaser or consumer. When the verified age of the prospective purchaser or consumer is equal to or greater than the minimum alcoholic beverage purchasing age the method 200 proceeds to 208. At 208, the intoxication level of the prospective purchaser or consumer is verified. At 210, it is determined whether the intoxication level of the prospective purchaser or consumer is less than an intoxication level threshold. When the intoxication level of the prospective purchaser or consumer is greater than or equal to the intoxication level threshold the method 200 denies selling, vending, and/or distributing alcoholic beverages to the prospective purchaser or consumer. In this manner, if the prospective purchaser or consumer fails to satisfy either the age requirement or the intoxication requirement, then the method 200 denies sales/vending of alcoholic beverages to that prospective purchaser or consumer. Referring again to 210, when the intoxication level of the prospective purchaser or consumer is less than the intoxication level the method 200 approves selling, vending, and/or distributing alcoholic beverages to the prospective purchaser or consumer. In other words, the prospective purchaser or consumer must satisfy both the age requirement and the intoxication requirement before the method 200 approves sales/vending of alcoholic beverages to that prospective purchaser or consumer. Moreover, upon the prospective purchaser or consumer satisfying both the age requirement and the intoxication requirement, the method 200 autonomously approves sales/vending of alcoholic beverages to that prospective purchaser or consumer. In an aspect, the steps of method 200 are performed by system 100. One of ordinary skill in the art will understand that verifying the intoxication level of the prospective purchaser or consumer at 208 and determining whether the intoxication level of the prospective purchaser or consumer is less than an intoxication level threshold at 210 may be performed before verifying the age of the prospective purchaser or consumer at 204 and determining whether the verified age of the prospective purchaser or consumer is equal to or greater than a minimum alcoholic beverage purchasing age at 206 without departing from the scope of the present invention.

FIG. 2B illustrates a method 220 of autonomously selling, vending, and/or distributing alcoholic beverages, according to another exemplary embodiment. In this embodiment, the selling, vending, and/or distributing of alcoholic beverages to the prospective purchaser or consumer is approved or denied based upon a verification of the age of the prospective purchaser or consumer.

FIG. 2C illustrates a method 230 of autonomously selling, vending, and/or distributing alcoholic beverages, according to another exemplary embodiment. In this embodiment, the selling, vending, and/or distributing of alcoholic beverages to the prospective purchaser or consumer is approved or denied based upon a verification of the intoxication level of the prospective purchaser or consumer.

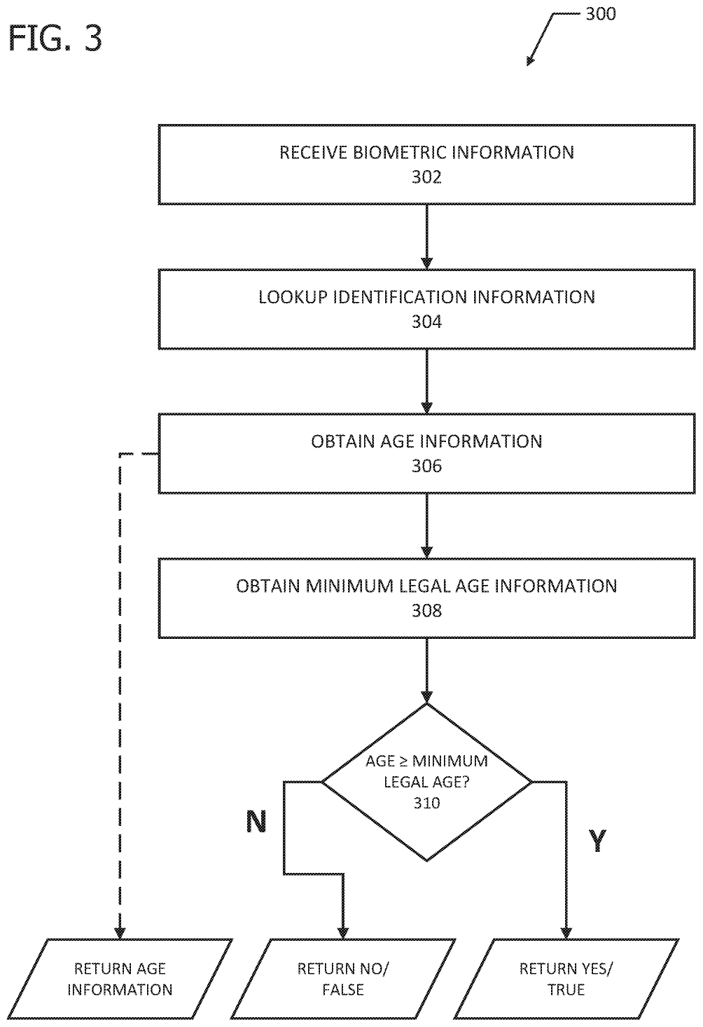

FIG. 3 illustrates a method 300 of autonomously verifying that a prospective purchaser or consumer of alcoholic beverages satisfies any applicable age restrictions. In an aspect, the steps of method 300 are performed by age verifier 104. At 302, information about physiological or behavioral characteristics of a prospective alcoholic beverage purchaser or consumer is received. The received information is used to lookup identification information of the prospective purchaser or consumer at 304. At 306, age information about the prospective purchaser or consumer is obtained from the identification information. At 308, information about minimum legal age restrictions on the purchase, sale, possession, and/or consumption of alcoholic beverages for a predefined legal jurisdiction is obtained. At 310, it is determined whether the age of the prospective purchaser or consumer is greater than or equal to the minimum legal age. When the age of the prospective purchaser or consumer is less than the minimum legal age the method 300 outputs/returns an indication (e.g., NO, FALSE, etc.) that the prospective purchaser or consumer does not satisfy the minimum age requirement, which may be used to deny selling, vending, and/or distributing alcoholic beverages to the prospective purchaser or consumer. When the age of the prospective purchaser or consumer is greater than or equal to the minimum legal age the method 300 outputs/returns an indication (e.g., YES, TRUE, etc.) that the prospective purchaser or consumer satisfies the minimum age requirement, which may be used to continue with other legal restriction verifications (e.g., intoxication level verification) and/or at least partially approve selling, vending, and/or distributing alcoholic beverages to the prospective purchaser or consumer. Optionally, the age information obtained at 306 may be directly outputted/returned by method 300.

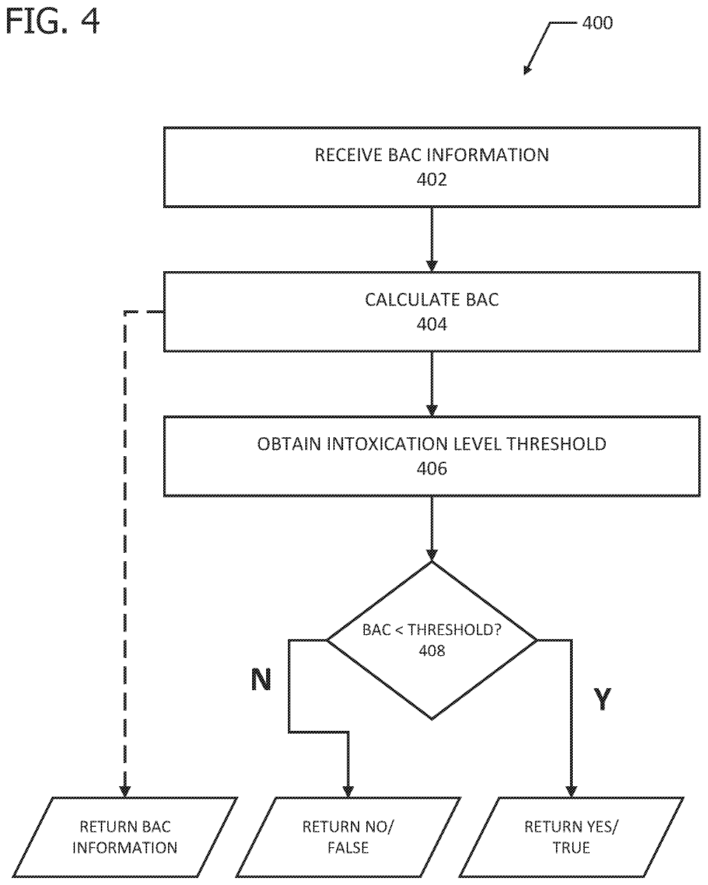

FIG. 4 illustrates a method 400 of autonomously verifying that a prospective purchaser or consumer of alcoholic beverages is not intoxicated beyond the acceptable limit. In an aspect, the steps of method 400 are performed by intoxication verifier 106. At 402, information about the blood alcohol content (BAC) of the prospective purchaser or consumer is received. The received information is used to calculate the BAC of the prospective purchaser or consumer at 404. At 406, information about the intoxication level threshold is obtained. For instance, the intoxication level threshold may be set to legal limits at which point operation of motor vehicles is prohibited (e.g., 0.08% in the United States) or may be altered depending upon other factors. At 408, it is determined whether the BAC of the prospective purchaser or consumer is less than the intoxication level threshold. When the BAC of the prospective purchaser or consumer is greater than or equal to the intoxication level threshold the method 400 outputs/returns an indication (e.g., NO, FALSE, etc.) that the prospective purchaser or consumer does not satisfy the intoxication threshold, which may be used to deny selling, vending, and/or distributing of alcoholic beverages to the prospective purchaser or consumer. When the BAC of the prospective purchaser or consumer is less than the intoxication level threshold the method 400 outputs/returns an indication (e.g., YES, TRUE, etc.) that the prospective purchaser or consumer satisfies the intoxication level requirement, which may be used to continue with other legal restriction verifications (e.g., age verification) and/or at least partially approve selling, vending, and/or distributing alcoholic beverages to the prospective purchaser or consumer. Optionally, the BAC of the prospective purchaser or consumer calculated at 404 may be directly outputted/returned by the method 400.

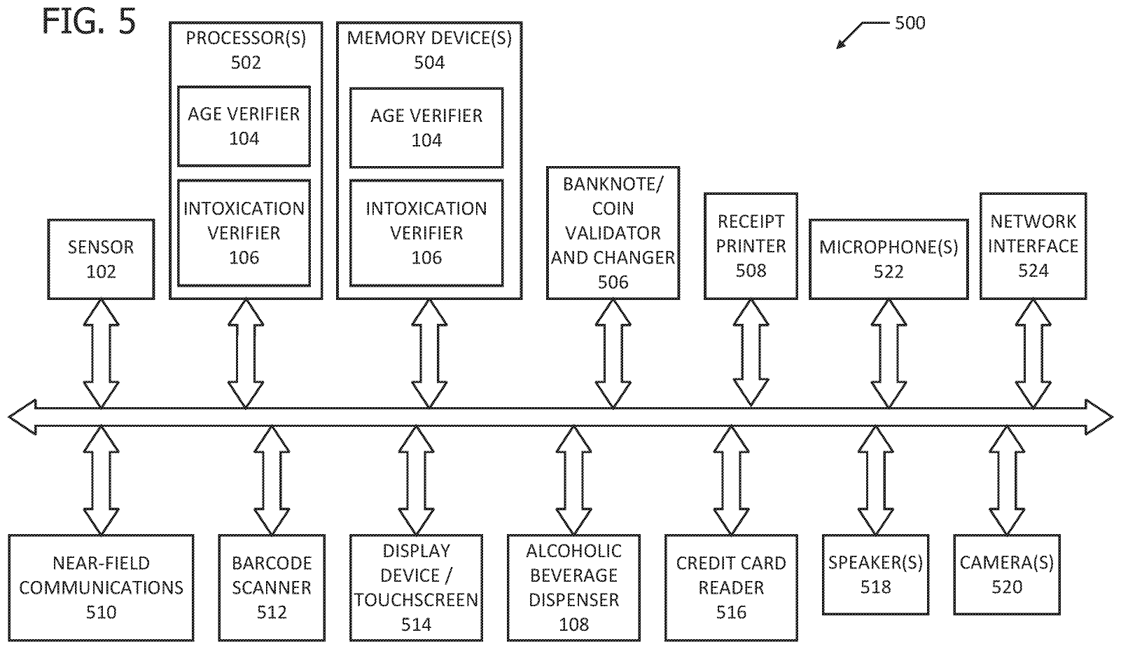

FIG. 5 is a block diagram of a system 500, according another exemplary embodiment. In this exemplary embodiment, the system 500 includes at least the sensor 102, the alcoholic beverage dispenser 108, one or more processors 502 (e.g., a central processing unit (CPU), a graphics processing unit (GPU) or both), one or more memory devices 504, a banknote/coin validator and changer 506, a receipt printer 508, a near-field communications (NFC) device 510, a barcode scanner 512, a display device/touchscreen device 514, a credit card reader 516, one or more speakers 518, one or more cameras 520, one or more microphones 522, and/or a network interface device 524, which communicate with each other via a bus 526. In this exemplary embodiment, the age verifier 104 and the intoxication verifier 106 are embodied, at least in part, as processor-executable instructions stored on the memory devices 504 and executed by the processors 502.

With reference to FIG. 5, an example embodiment extends to a machine in the example form of system 500 within which instructions for causing the machine to perform any one or more of the methodologies discussed herein may be executed. In alternative example embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. Further, while only a single machine is illustrated, the term “machine” shall also be taken to include any collection of machines that individually or jointly execute a set (or multiple sets) of instructions to perform any one or more of the methodologies discussed herein.

The memory devices 504 include a machine-readable medium on which is stored one or more sets of instructions and data structures (e.g., software instructions) embodying or used by any one or more of the methodologies or functions described herein. The instructions may also reside, completely or at least partially, within the main memory 504 or within the processor 502 during execution thereof by the system 500, the main memory 504 and the processor 502 also constituting machine-readable media.

While the machine-readable medium is described in an example embodiment to be a single medium, the term “machine-readable medium” may include a single medium or multiple media (e.g., a centralized or distributed database, or associated caches and servers) that store the one or more instructions. The term “machine-readable medium” shall also be taken to include any tangible medium that is capable of storing, encoding, or carrying instructions for execution by the machine and that cause the machine to perform any one or more of the methodologies of embodiments of the present invention, or that is capable of storing, encoding, or carrying data structures used by or associated with such instructions. The term “machine-readable storage medium” shall accordingly be taken to include, but not be limited to, solid-state memories and optical and magnetic media that can store information in a non-transitory manner, i.e., media that is able to store information. Specific examples of machine-readable media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks.

The instructions may further be transmitted or received over a communications network using a signal transmission medium via the network interface device 524 and utilizing any one of a number of well-known transfer protocols (e.g., FTP, HTTP). Examples of communication networks include a local area network (LAN), a wide area network (WAN), the Internet, mobile telephone networks, Plain Old Telephone (POTS) networks, wireless data networks (e.g., WiFi and WiMax networks), and the like, including combinations thereof. The term “machine-readable signal medium” shall be taken to include any transitory intangible medium that is capable of storing, encoding, or carrying instructions for execution by the machine, and includes digital or analog.

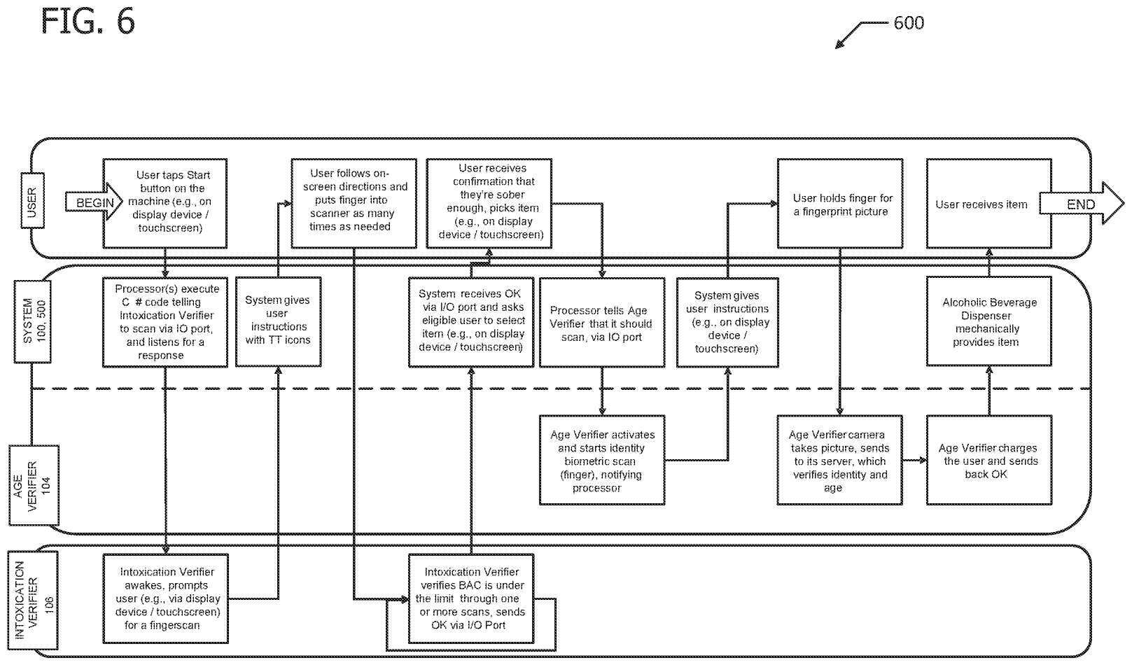

FIG. 6 illustrates an architecture and method 600 of autonomously selling, vending, and/or distributing alcoholic beverages, according to another exemplary embodiment.

Thus, methods and systems for autonomous selling, vending, and/or distributing of alcoholic beverages upon autonomous verification that the prospective purchaser or consumer satisfies any applicable age restrictions and is not intoxicated beyond the acceptable limit have been described. Although embodiments of the present invention have been described with reference to specific example embodiments, it will be evident that various modifications and changes may be made to these embodiments without departing from the broader spirit and scope of the embodiments of the invention. Accordingly, the specification and drawings are to be regarded in an illustrative rather than a restrictive sense.

The methods described herein do not have to be executed in the order described, or in any particular order. Moreover, various activities described with respect to the methods identified herein can be executed in serial or parallel fashion. Although “End” blocks are shown in the flowcharts, the methods may be performed continuously.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A system, comprising:
a sensor, wherein the sensor is operable to receive biometric information of a prospective alcoholic beverage purchaser or consumer;
an age verifier, wherein the age verifier is operable to:
autonomously determine an age of the prospective purchaser or consumer based at least in part upon the received biometric information, and
autonomously determine whether the determined age of the prospective purchaser or consumer satisfies a minimum alcoholic beverage purchasing age; and
an alcoholic beverage dispenser, wherein the alcoholic beverage dispenser is operable to autonomously vend or dispense one or more alcoholic beverages to the purchaser or consumer based at least in part upon autonomously determining that the prospective purchaser or consumer satisfies the minimum alcoholic beverage purchasing age.

2. The system of claim 1, wherein the sensor is a fingerprint reader and the biometric information includes, at least in part, a fingerprint of the prospective purchaser or consumer.

3. The system of claim 1,
wherein the sensor is operable to receive blood alcohol content (BAC) information of the prospective purchaser or consumer,
the system further comprising an intoxication verifier, wherein the intoxication verifier is operable to:
autonomously determine an intoxication level of the prospective purchaser or consumer based at least in part upon the received BAC information, and
autonomously determine whether the determined intoxication level satisfies an intoxication level threshold; and
wherein the alcoholic beverage dispenser is operable to autonomously vend or dispense one or more alcoholic beverages to the purchaser or consumer based at least in part upon autonomously determining that the prospective purchaser or consumer satisfies both the minimum alcoholic beverage purchasing age and the intoxication level threshold.

4. The system of claim 3, wherein the sensor is operable to receive the BAC information of the prospective purchaser or consumer by spectroscopically scanning a finger of the prospective purchaser or consumer.

5. The system of claim 3,
wherein the sensor is comprised of a fingerprint reader, a light source, an interferometer, and a detector,
wherein the light source is operable to illuminate the skin of a finger of the prospective purchaser or consumer,
wherein the interferometer is operable to filter light reflected by the skin of the finger of the prospective purchaser or consumer for wavelengths relevant to blood alcohol content measurement, and
wherein the detector is operable to analyze the filtered light and report the BAC level of the prospective purchaser or consumer to the intoxication verifier.

6. The system of claim 3, further comprising a network interface device, wherein the network interface device is operable to communicatively coupled the age verifier to an identity database including one or more biometric profiles attaching an identity of the prospective purchaser or consumer to a fingerprint of the prospective purchaser or consumer.

7. The system of claim 5 further comprising:
a processor;
a memory device;
a touchscreen display device;
at least one of a near-field communications device, a barcode scanner, and a camera; and
at least one of a credit card reader and a banknote/coin validator and changer.

8. A system, comprising:
a sensor, wherein the sensor is operable to receive at least one of:
biometric information of a prospective alcoholic beverage purchaser or consumer, and
blood alcohol content (BAC) information of the prospective purchaser or consumer;
an age verifier, wherein the age verifier is operable to:
autonomously determine an age of the prospective purchaser or consumer based at least in part upon the received biometric information, and
autonomously determine whether the determined age of the prospective purchaser or consumer satisfies a minimum alcoholic beverage purchasing age;
an intoxication verifier, wherein the intoxication verifier is operable to:
autonomously determine an intoxication level of the prospective purchaser or consumer based at least in part upon the received BAC information, and
autonomously determine whether the determined intoxication level satisfies an intoxication level threshold; and
an alcoholic beverage dispenser, wherein the alcoholic beverage dispenser is operable to autonomously vend or dispense one or more alcoholic beverages to the purchaser or consumer based at least in part upon autonomously determining that the prospective purchaser or consumer satisfies either the minimum alcoholic beverage purchasing age or the intoxication level threshold.

9. The system of claim 8, wherein the sensor is a fingerprint reader and the biometric information includes, at least in part, a fingerprint of the prospective purchaser or consumer.

10. The system of claim 8, wherein the sensor is operable to receive the BAC information of the prospective purchaser or consumer by spectroscopically scanning a finger of the prospective purchaser or consumer.

11. The system of claim 8,
wherein the sensor is comprised of a fingerprint reader, a light source, an interferometer, and a detector,
wherein the light source is operable to illuminate the skin of a finger of the prospective purchaser or consumer,
wherein the interferometer is operable to filter light reflected by the skin of the finger of the prospective purchaser or consumer for wavelengths relevant to blood alcohol content measurement, and
wherein the detector is operable to analyze the filtered light and report the BAC level of the prospective purchaser or consumer to the intoxication verifier.

12. The system of claim 8, further comprising a network interface device, wherein the network interface device is operable to communicatively coupled the age verifier to an identity database including one or more biometric profiles attaching an identity of the prospective purchaser or consumer to a fingerprint of the prospective purchaser or consumer.

13. The system of claim 12 further comprising:
a processor;
a memory device;
a touchscreen display device;
at least one of a near-field communications device, a barcode scanner, and a camera; and
at least one of a credit card reader and a banknote/coin validator and changer.

14. A method, comprising:
receiving, by a sensor, biometric information of a prospective alcoholic beverage purchaser or consumer;
autonomously determining, by an age verifier, an age of the prospective purchaser or consumer based at least in part upon the received biometric information;
autonomously determining, by the age verifier, whether the determined age of the prospective purchaser or consumer satisfies a minimum alcoholic beverage purchasing age; and
vending or dispensing one or more alcoholic beverages to the purchaser or consumer based at least in part upon autonomously determining that the prospective purchaser or consumer satisfies the minimum alcoholic beverage purchasing age.

15. The method of claim 14, wherein the sensor is a fingerprint reader and the biometric information includes, at least in part, a fingerprint of the prospective purchaser or consumer.

16. The method of claim 14, further comprising:
receiving, by the sensor, blood alcohol content (BAC) information of the prospective purchaser or consumer;
autonomously determining, by an intoxication verifier, an intoxication level of the prospective purchaser or consumer based at least in part upon the received BAC information;
autonomously determining, by the intoxication verifier, whether the determined intoxication level satisfies an intoxication level threshold; and
vending or dispensing the one or more alcoholic beverages to the purchaser or consumer based at least in part upon autonomously determining that the prospective purchaser or consumer satisfies both the minimum alcoholic beverage purchasing age and the intoxication level threshold.

17. The method of claim 16, wherein said receiving, by the sensor, the BAC information of the prospective purchaser or consumer includes spectroscopically scanning a finger of the prospective purchaser or consumer.

18. The method of claim 16,
wherein the sensor is comprised of a fingerprint reader, a light source, an interferometer, and a detector, and
the method further comprising:
illuminating, by the light source, the skin of a finger of the prospective purchaser or consumer;
filtering, by the interferometer, light reflected by the skin of the finger of the prospective purchaser or consumer for wavelengths relevant to blood alcohol content measurement;
analyzing, by the detector, the filtered light; and
reporting, by the detector, the BAC level of the prospective purchaser or consumer to the intoxication verifier.

19. The method of claim 18, wherein the biometric information includes, at least in part, a fingerprint of the prospective purchaser or consumer.

20. The method of claim 19, further comprising communicating, via a network interface device, with an identity database including one or more biometric profiles attaching an identity of the prospective purchaser or consumer to a fingerprint of the prospective purchaser or consumer.
